(19) United States
(12) Patent Application Publication
(10) Pub. No.: US 2016/0164519 A1
(43) Pub. Date: Jun. 9, 2016

(54) TOUCHLESS ON/OFF TATTOO COMPONENT FOR HANDHELD TATTOO MACHINES AND TATTOO POWER SUPPLIES

(71) Applicant: Finger Wave, LLC

(72) Inventor: Amory Roberto Arriaga, Tucson, AZ (US)

(73) Assignee: Finger Wave, LLC, Tucson, AZ (US)

(21) Appl. No.: 14/956,378

(22) Filed: Dec. 1, 2015

Related U.S. Application Data
(60) Provisional application No. 62/123,942, filed on Dec. 3, 2014.

(51) Int. Cl. H03K 17/945; A61M 37/00

(52) U.S. Cl. CPC H03K 17/945; A61M 37/0076

(57) ABSTRACT

The invention consists of a touchless on/off tattoo component that manipulates the operation of hand held tattoo machines and tattoo power supplies through the use of motion with any body part; such as but not limited to a finger, fingers, hand, hands, arm and arms. The touchless on/off tattoo component includes a housing for retaining the interior electronic components needed to operate a hand held tattoo machine and a tattoo power supply. A sensor enables touchless activation through the use of motion with any body part across the face of the sensor; as well as deactivation with the use of the same motion.

### CROSS-REFERENCE TO RELATED APPLICATIONS

This application claims the priority benefit of U.S. Provisional Patent Application Ser. No. 62/123,942 filed Dec. 3, 2014.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### REFERENCE TO A “SEQUENCE LISTING,” A TABLE, OR A COMPUTER PROGRAM LISTING COMPACT DISK APPENDIX

Not Applicable

### BACKGROUND OF THE INVENTION

(1) Field of the Invention

The invention relates to hand held tattoo machines, tattoo power supplies and methods of operation. More particularly, the invention relates to a touchless on/off tattoo component that manipulates the operation of a hand held tattoo machine and tattoo power supplies through the use of motion with any body part; such as but not limited to a finger, fingers, hand, hands, arm and arms.

(2) Description of the Related Art

A tattoo artist manipulates a hand held tattoo machine to produce a tattoo. A hand held tattoo machine is controlled and powered by a tattoo power supply. A hand held tattoo machine and power supply combined are turned on and off by a foot switch operated by foot or turned on and off with a toggle switch operated by a finger.

Most states have determined that the tattoo industry is a hazardous practice due to the utilization of needles puncturing a client's skin during the application of the tattoo. The potential of disease is such a concern in some states that these states require wearing single use gloves and properly wrapping all surfaces and equipment handled.

The vast majority of tattoo artists use foot pedals and switches to operate the on and off control of the tattoo machines and power supplies, which are physically handled throughout the entire tattoo. The constant physical use of the foot pedal and toggle switch is one of the short comings in regards to the execution of sanitation.

### BRIEF SUMMARY OF THE INVENTION

A touchless on/off component (55) comprises of a housing (54) for interior electronic components (35) for operating a hand held tattoo-machine (50) and a tattoo power supply (38). A sensor (34) is mounted to the housing (54) and the interior electronic components (35). The sensor (34) enables touchless activation of the hand held tattoo machine (50) and the tattoo power supply (38) through the use of motion with any body part across the face of the sensor (34) as well as deactivation with the use of the same motion.

### BRIEF DESCRIPTION OF THE SEVERAL VIEWS OF THE DRAWING

FIG. 1 is a three quarter view of the touchless on/off tattoo component (55), the power supply cable (26) and the wall power cable (28), shown with the power source connector (24) inserted into the power source jack (20) and with the power supply connector (22) inserted into the power supply jack (18).

FIG. 2 is a three quarter view of the touchless on/off tattoo component (55), shown with the power source connector (24) disconnected from the power source jack (20) and with the power supply connector (22) disconnected from the power supply jack (18).

FIG. 3 is a front view of the touchless on/off tattoo component (55).

FIG. 4 is a side view of the touchless on/off tattoo component (55).

FIG. 5 is a perspective view of the touchless on/off tattoo component (55) being controlled with a hand motion.

FIG. 6 is a perspective view of the touchless on/off tattoo component connected to a power source (52), tattoo power supply (38), and hand held tattoo machine (50) through a power supply cable (26), wall power cable (28) and a tattoo machine cable (46).

### DETAILED DESCRIPTION OF THE INVENTION

A touchless on/off component (55) comprises of a housing (54) for interior electronic components (35) for operating a hand held tattoo machine (50) and a tattoo power supply (38).

The housing (54) of the touchless on/off component (55) includes a top wall (8), a front wall (10), a first side wall (12A) and a second side wall (12B), a back wall (14) and a bottom wall (16).

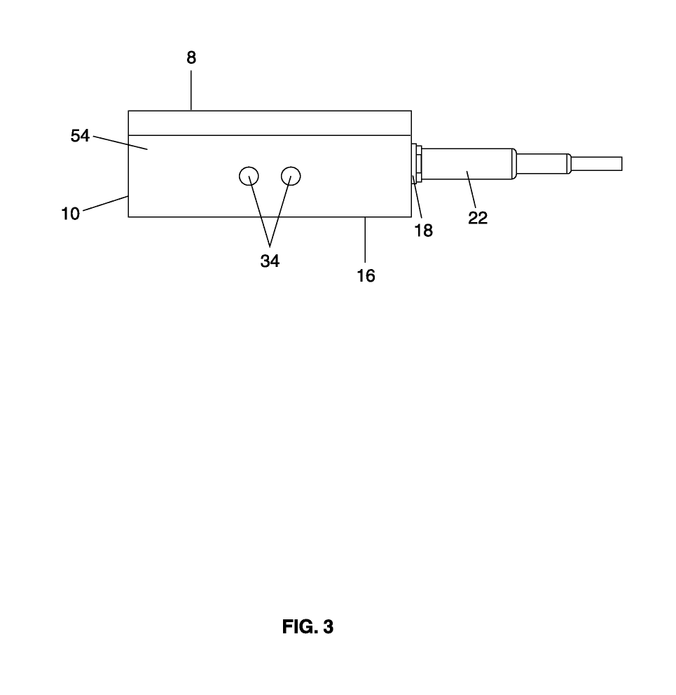

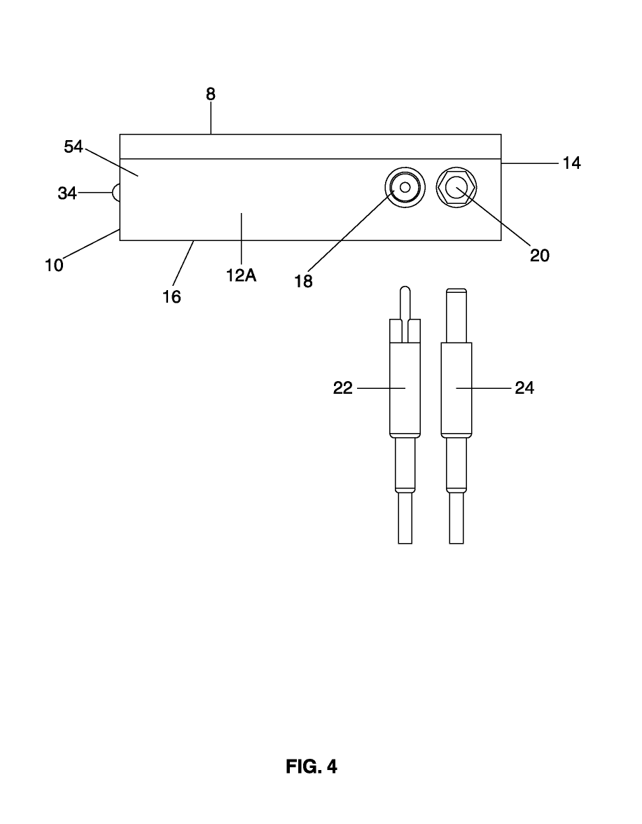

A power supply jack (18) is mounted on the first side wall (12A) and interior components (35). A power source jack (20) is mounted to the first side wall (12A) and interior electronic components (35). A sensor (34) is mounted to the front wall (10) and interior electronic components (35). FIG. 3 is a front view of the touchless on/off tattoo component (55). FIG. 4 is a side view of the touchless on/off tattoo component (55).

If the touchless on/off component (55) is on, when an object, such as a hand, is moved across the sensor (34), the sensor (34) detects this movement and through the interior electronic components (35), turns on the touchless on/off component (55); the sensor (34) and the interior electronic components (35) act as a power switch. If the touchless on/off component (55) is off, the touchless on/off component (55) will turn on if an object is moved across the sensor (34). When the touchless on/off component (55) is on, the touchless on/off component (55) supplies power to the tattoo power supply (38) through the power supply jack (18).

This touchless on/off component (55) is used with various other components to provide the tattoo artist with a tattoo device capable of tattooing without having the tattoo artist touch the device. As mentioned above, this capability provides substantial sanitary benefits for the tattooing process.

A power source connector (24) is plugged into the power source jack (20). A first end of a wall power cable (28) is attached to the power source connector (24). A second end of the wall power cable (28) is attached to a power plug (32). A power supply connector (22) is plugged into the power supply jack (18). A first end of a power supply cable (26) is attached to the power supply connector (22). A second end of the power supply cable (26) is attached to a power-in connector (30).

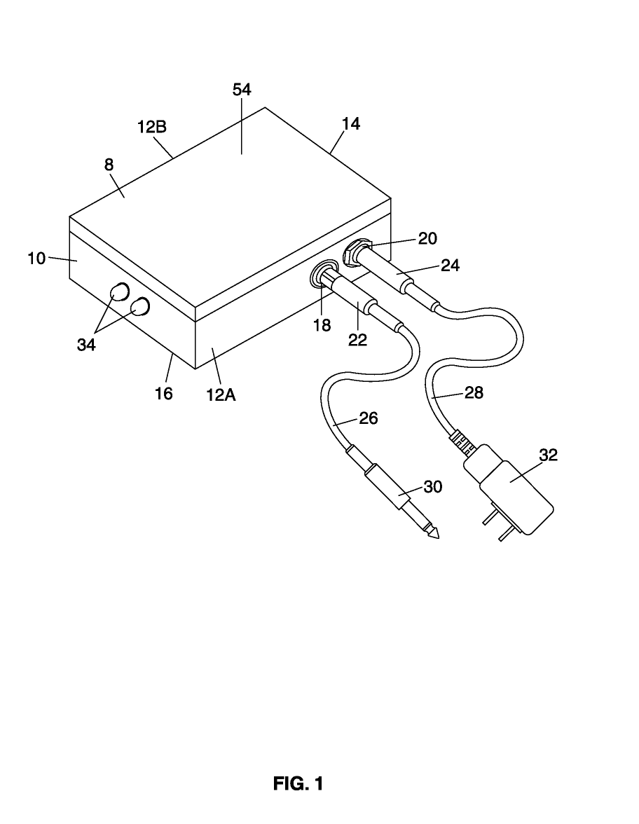

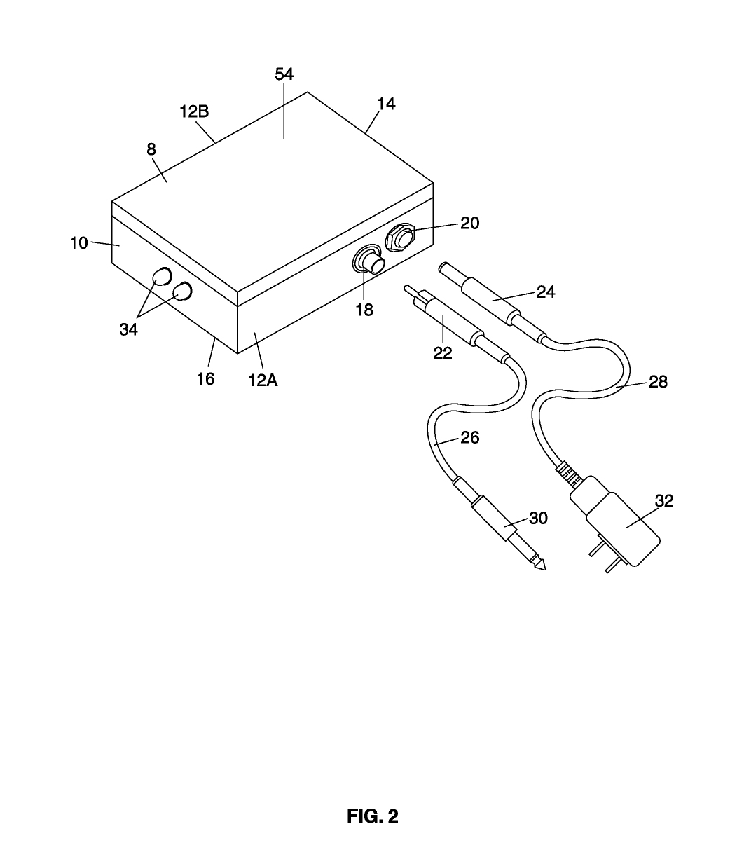

FIG. 1 is a three quarter view of the touchless on/off tattoo component (55), the power supply cable (26) and the wall power cable (28), shown with the power source connector (24) inserted into the power source jack (20) and with the power supply connector (22) inserted into the power supply jack (18). FIG. 2 is a three quarter view of the touchless on/off tattoo component (55), shown with the power source connector (24) disconnected from the power source jack (20) and with the power supply connector (22) disconnected from the power supply jack (18).

A tattoo power supply (38) provides power to the hand held tattoo machine (50). The tattoo power supply (38) has a power-in jack (40) and a power-out jack (42). The power-in connector (30) is plugged to the power-in jack (40).

A power-out connector (44) is plugged into the power-out jack (42). A first end of a tattoo machine cable (46) is attached to the power-out connector (44). A second end of the tattoo machine cable (46) is attached to a tattoo machine. attachment (48).

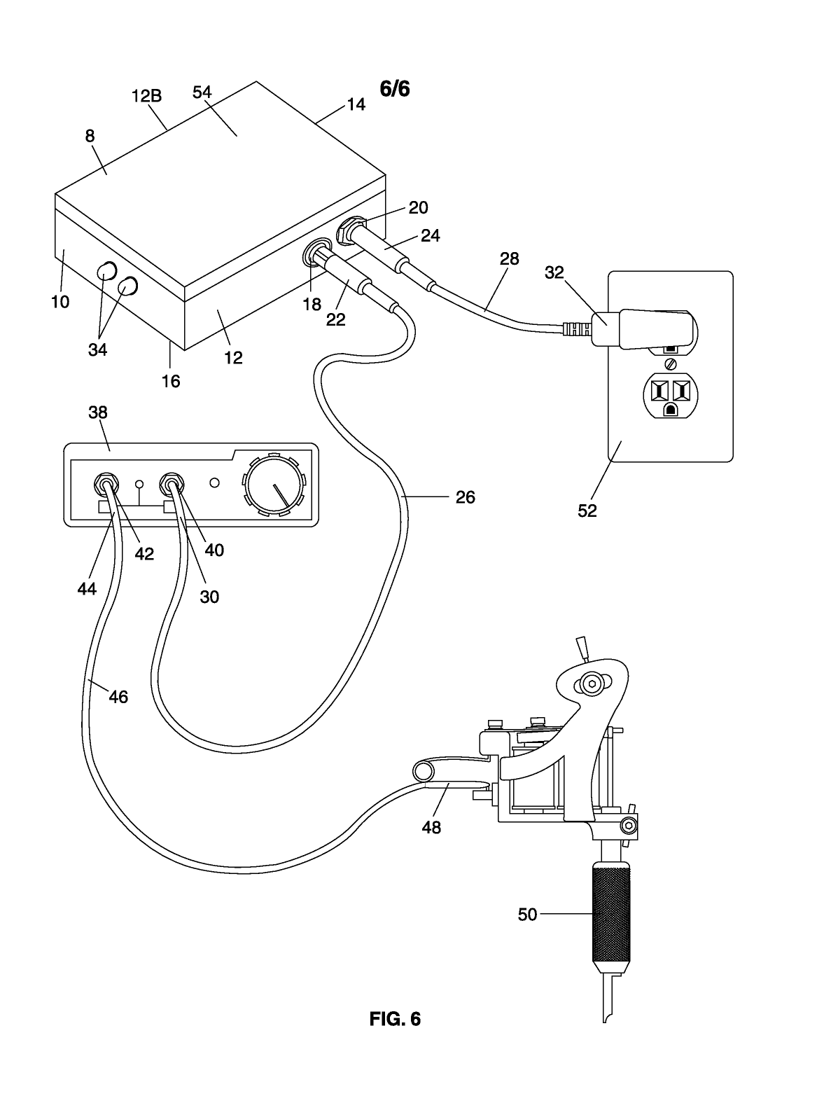

The tattoo machine attachment (48) is attached to the hand held tattoo machine (50). The hand held tattoo machine (50) is used to produce tattoos. FIG. 6 is a perspective view of the touchless on/off tattoo component connected to a power source (52), tattoo power supply (38), and hand held tattoo machine (50) through a power supply cable (26), wall power cable (28) and a tattoo machine cable (46).

To set up the tattoo device, the following steps are performed in no specific order. Insert power plug (32) to a power source (52). Insert power source connector (24) into power source jack (20). Insert power supply connector (22) into power supply jack (18). Insert power-in connector (30) into power-in jack (40). Insert power-out connector (44) to power-out jack (42). Attach tattoo machine attachment (48) to hand held tattoo machine (50). FIG. 6 shows how the various components are connected after the tattoo device is set-up.

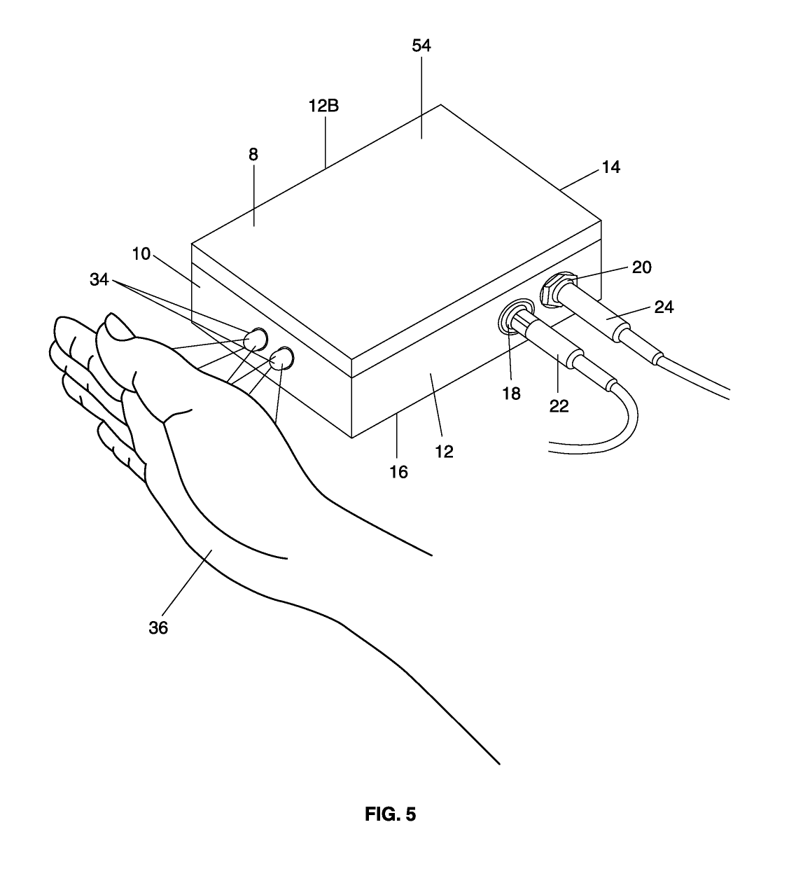

In operation the tattoo artist moves his hand (36) across the sensor (34) on the front wall (10) on the touchless on/off tattoo component (55) as shown in FIG. 5. Doing so will turn the touchless on/off component (55) on which will activate the tattoo power supply (38) to give power to the hand held tattoo machine (50). The artist can then make a motion of his hand (36) across the sensor (34) to turn the touchless on/off tattoo component (55) off.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

I claim:

1. A device for producing tattoos, which comprises:
a.) a touchless on/off component, which comprises
i.) a housing;
ii.) a sensor, where the sensor is mounted on the housing;
iii.) interior electronic components, where the sensor is mounted on the interior electronic components;
iv.) a power source jack, where the power source jack is mounted on the housing;
v.) a power supply jack, where the power supply jack is mounted on the housing.

2. The apparatus in claim 1, wherein the housing further comprises:
a.) a top wall;
b.) a bottom wall;
c.) a first side wall, where the power source jack is mounted on the first side wall and the power supply jack is mounted on the first side wall;
d.) a second side wall;
e.) a front wall, where the sensor is mounted on the front wall;
f.) a back wall.

3. A method for producing tattoos using the apparatus in claim 2, which comprises the steps of
a.) Moving a hand across the sensor to turn on the touchless on/off component when the touchless on/off component is turned off;
b.) Moving the hand across the sensor to turn off the touchless on/off component when the touchless on/off component is turned on.
